(19) United States
(12) Patent Application Publication
(10) Pub. No.: US 2007/0292890 A1
(43) Pub. Date: Dec. 20, 2007

(54) Chemiluminescence chip and chemiluminescence detecting apparatus using the same

(76) Inventors: Osamu Nozaki, Osakasayama-shi (JP); Motonori Munesue, Matsubara-shi (JP); Hiroko Kawamoto, Yonago-shi (JP)

Correspondence Address: FLYNN THIEL BOUTELL & TANIS, P.C., 2026 RAMBLING ROAD, KALAMAZOO, MI, 49008-1631, US

(21) Appl. No.: 11/455,474

(22) Filed: Jun. 19, 2006

(51) Int. Cl. G01N 33/53; C12M 3/00

(52) U.S. Cl. 435/7.1; 435/287.2

(57) ABSTRACT

Disclosed herein are a chemiluminescence chip, which is used to detect and quantify various kinds of materials in the case of performing the enzyme labeling of a material to be measured and performing the chemiluminescence measurement of the activity of the labeled enzyme, and a chemiluminescence detecting apparatus using the same. The chemiluminescence chip is constructed such that a filling unit, in which an immobilized enzyme is filled, is mounted in a space having an inlet port, through which a material to be measured is introduced, and an outlet port, through which the material to be measured is discharged, on a supporting structure. The chemiluminescence detecting apparatus is constructed such that the above-described chemiluminescence chip is mounted in a dark box, a cooled CCD camera is mounted above the chemiluminescence chip, an inlet pipe, which is disposed outside the dark box, is connected to the inlet port of the filling unit of the chemiluminescence chip, and an outlet pipe, which is also disposed outside the dark box, is connected to the outlet port of the filling unit.

### BACKGROUND OF THE INVENTION

1. Field of the Invention

The present invention relates to a chemiluminescence chip used to detect and quantify various kinds of materials, for example, in a clinical testing field, and a chemiluminescence detecting apparatus using the same.

2. Description of the Related Art

From the past, there have been developed methods of performing the enzyme labeling of a material to be measured and performing the chemiluminescence measurement of the activity of the labeled enzyme in a clinical testing field. The chemiluminescence measuring methods are suitable in the clinical test because of the convenience, the promptness, and the high sensitivity thereof. The enzyme used in the chemiluminescence measuring method may be peroxidase, alkaliphosphatase, or glucose oxidase. The chemiluminescence material used in the chemiluminescence measuring method may be luminol, isoluminol, lophine, lucigenin, or peracetate (Patent Documents 1 to 6).

Also, the inventors of the present invention have invented a method of measuring hydrogen peroxide in a liquid, which is necessary to measure oxidative stress and the substrate of oxidase enzyme in a clinical testing field (for example, a method of measuring glucose or cholesterol), i.e., a method of measuring the strength of chemiluminescence generated by reaction of horseradish peroxidase, hydrogen peroxide, and imidazole (Patent Document 7).

[Patent Document 1] Japanese Unexamined Patent Publication No. 59-171839

[Patent Document 2] Japanese Unexamined Patent Publication No. 2-174694

[Patent Document 3] Japanese Unexamined Patent Publication No. 2-291299

[Patent Document 4] Japanese Unexamined Patent Publication No. 3-35147

[Patent Document 5] Japanese Unexamined Patent Publication No. 7-327694

[Patent Document 6] Japanese Unexamined Patent Publication No. 8-313443

[Patent Document 7] Japanese Unexamined Patent Publication No. 2004-81138

However, it is impossible to show an image by flow injection chemiluminescence detecting apparatuses using photomultiplier tubes even when any of the above-described chemiluminescence measuring methods is used. Showing the detection of a material to be measured in the form of an image is important for a person that estimates and diagnoses the detection of the material to be measured to be strongly influenced by the image and to more clearly understand the detection of the material to be measured.

### SUMMARY OF THE INVENTION

Therefore, the present invention has been made in view of the above problems, and it is an object of the present invention to provide a chemiluminescence chip used to detect and quantify various kinds of materials in the case of performing the enzyme labeling of a material to be measured and performing the chemiluminescence measurement of the activity of the labeled enzyme in a clinical testing field, and a chemiluminescence detecting apparatus using the same.

In accordance with one aspect of the present invention, the above and other objects can be accomplished by the provision of a chemiluminescence chip constructed such that a filling unit, in which an immobilized enzyme is filled, is mounted in a space having an inlet port, through which a material to be measured is introduced, and an outlet port, through which the material to be measured is discharged, on a supporting structure.

In the chemiluminescence chip according to the present invention, the filling unit may be a transparent tube, a slit formed in a synthetic resin sheet, or a groove formed in a flat plate.

Also, in the chemiluminescence chip according to the present invention, the filling unit is formed in the shape of a straight line, a zigzag, or a serpentine.

In accordance with another aspect of the present invention, there is provided a chemiluminescence detecting apparatus constructed such that the above-described chemiluminescence chip is mounted in a dark box, a cooled charge coupled device (CCD) camera is mounted above the chemiluminescence chip, an inlet pipe, which is disposed outside the dark box, is connected to the inlet port of the filling unit of the chemiluminescence chip, and an outlet pipe, which is also disposed outside the dark box, is connected to the outlet port of the filling unit.

In the chemiluminescence detecting apparatus according to the present invention, the dark box has a heater and a temperature sensor mounted therein.

The chemiluminescence chip according to the present invention has an effect in that the reaction between the material to be measured and the labeled enzyme can be rapidly and conveniently carried out. Also, the chemiluminescence detecting apparatus using the chemiluminescence chip according to the present invention has an effect in that the detection and quantification of the various kinds of materials can be easily carried out.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a plan view illustrating a chemiluminescence chip according to a preferred embodiment of the present invention;

FIG. 2 is a side view of the chemiluminescence chip according to the present invention shown in FIG. 1;

FIG. 3 is a plan view illustrating a chemiluminescence chip according to another preferred embodiment of the present invention;

FIG. 4 is a side view of the chemiluminescence chip according to the present invention shown in FIG. 3;

FIG. 5 is a plan view illustrating a chemiluminescence chip according to another preferred embodiment of the present invention;

FIG. 6 is a side view of the chemiluminescence chip according to the present invention shown in FIG. 5;

FIG. 7 is a plan view illustrating a chemiluminescence chip according to yet another preferred embodiment of the present invention;

FIG. 8 is a side view, partly in section, of the chemiluminescence chip according to the present invention shown in FIG. 7;

FIG. 9 is a sectional view schematically illustrating a chemiluminescence detecting apparatus according to the present invention; and

FIG. 10 is a block diagram schematically illustrating the construction of the chemiluminescence detecting apparatus according to the present invention.

### DESCRIPTION OF THE PREFERRED EMBODIMENTS

Now, a chemiluminescence chip according to the present invention and a chemiluminescence detecting apparatus using the same will be described in detail with reference to the accompanying drawings.

As shown in the drawings, the chemiluminescence chip according to the present invention is constructed such that a filling unit 1, in which an immobilized enzyme 2 is filled, is mounted in a space having an inlet port 1a, through which a material to be measured is introduced, and an outlet port 1b, through which the material to be measured is discharged, on a supporting structure 4.

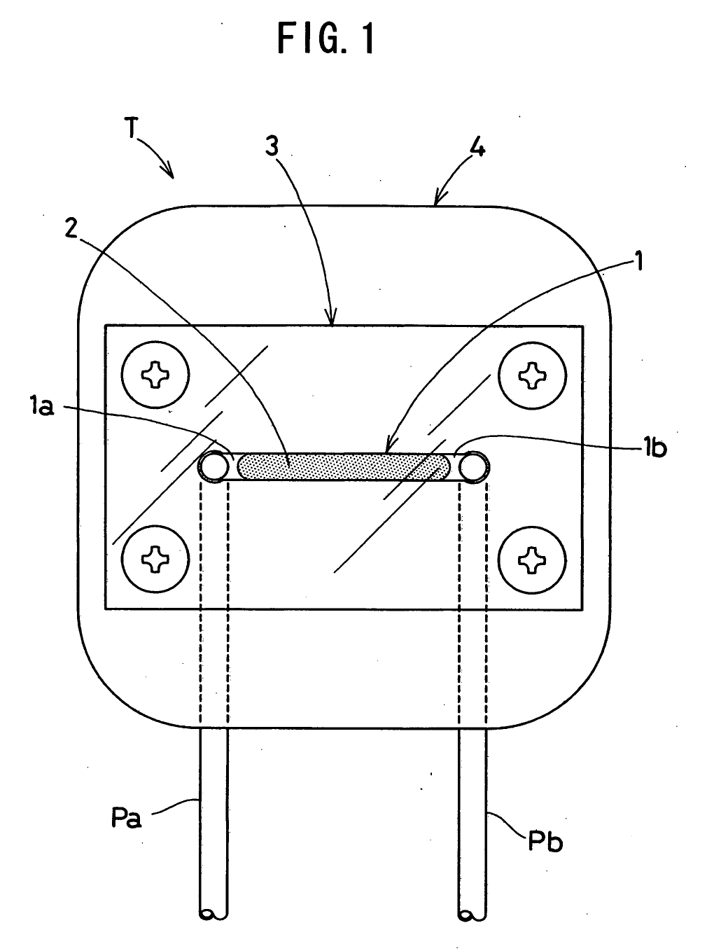

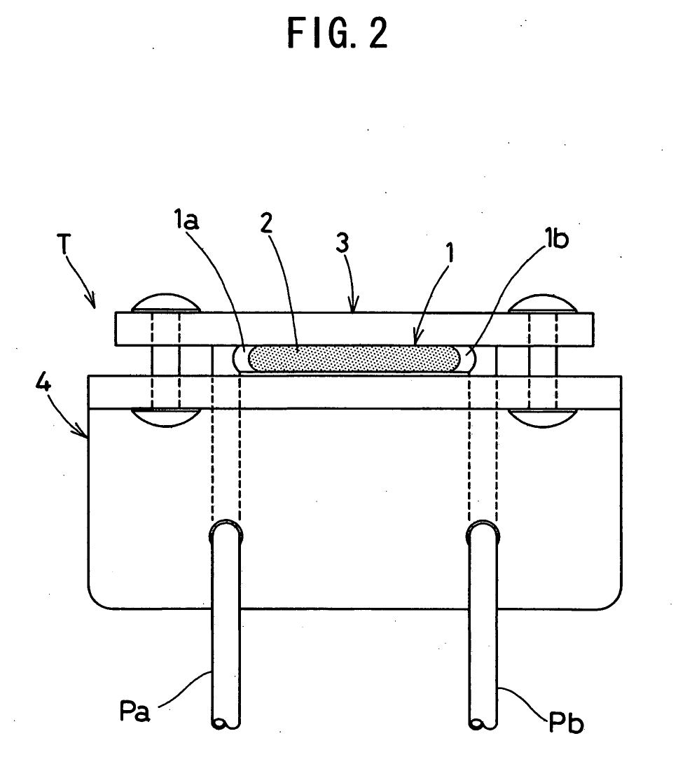

In the chemiluminescence chip according to the present invention shown in FIGS. 1 to 6, the filling unit 1 is a transparent tube, and the transparent tube is pressed against the supporting structure 4 by a transparent plate 3.

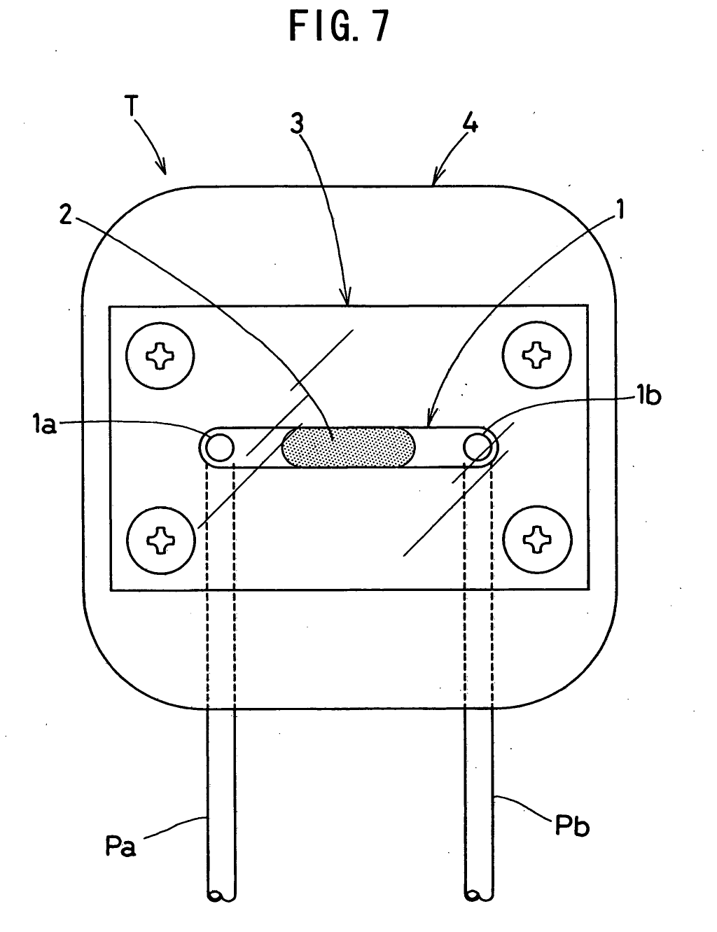

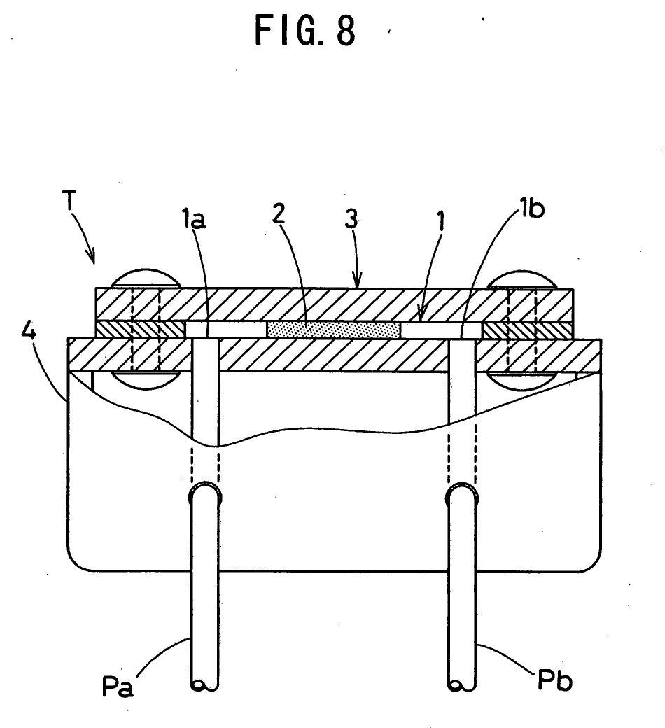

In the chemiluminescence chip according to the present invention shown in FIGS. 7 and 8, the filling unit 1 is a slit formed in a synthetic resin sheet, and the synthetic resin sheet is pressed against the supporting structure 4 by the transparent plate 3.

Although not shown in the drawings, the chemiluminescence chip according to the present invention may be constructed such that the filling unit 1 is a groove formed in a flat plate, and the flat board is pressed against the supporting structure 4 by the transparent plate 3.

In the case that the filling unit 1 is formed of the transparent tube, it is preferable to use a tube having high chemical resistance and high translucency, such as a Teflon tube, as the transparent tube.

In the case that the filling unit 1 is formed of the synthetic resin sheet, it is preferable to use a sheet having high chemical resistance, such as a Teflon sheet, as the synthetic resin sheet.

In the case that the filling unit 1 is formed of the flat plate, it is preferable to use a synthetic resin plate or a metal plate having high chemical resistance, such as a Teflon sheet, as the flat plate. Also, the groove is formed in the flat plate by a cutting process or an optical etching process.

It is also preferable to use a plate having high chemical resistance and high translucency, such as a Teflon plate or a glass plate, as the transparent plate 3.

In the chemiluminescence chip according to the present invention, hydrogen peroxide, a compound having an imidazole ring, or an oxidizing enzyme having hydrogen peroxide generated therein may be used as the material to be measured. However, the material to be measured is not restricted to the above-specified materials.

Also, in the chemiluminescence chip according to the present invention, peroxidase, alkaliphosphatase, or glucose oxidase may be used as the above-mentioned enzyme. However, the enzyme usable in the present invention is not restricted to the above-specified enzymes. In addition, the immobilized phase of the immobilized enzyme 2 may be gel having amino group, such as kitosan gel, glass beads, polystyrene gel, or acryl gel. However, the immobilized phase of the immobilized enzyme 2 is not restricted to the above-specified immobilized phases.

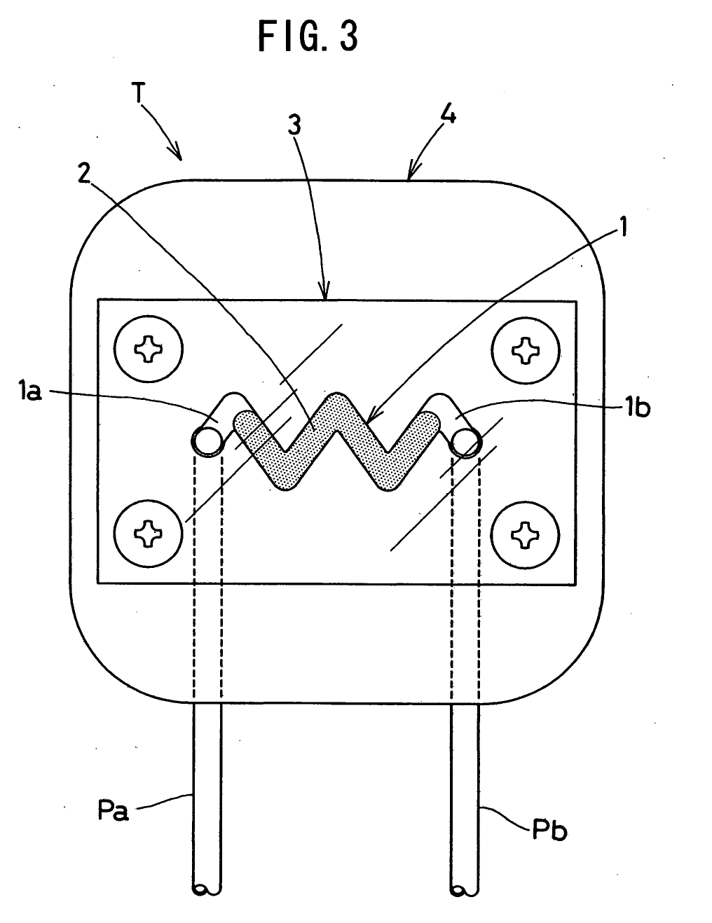

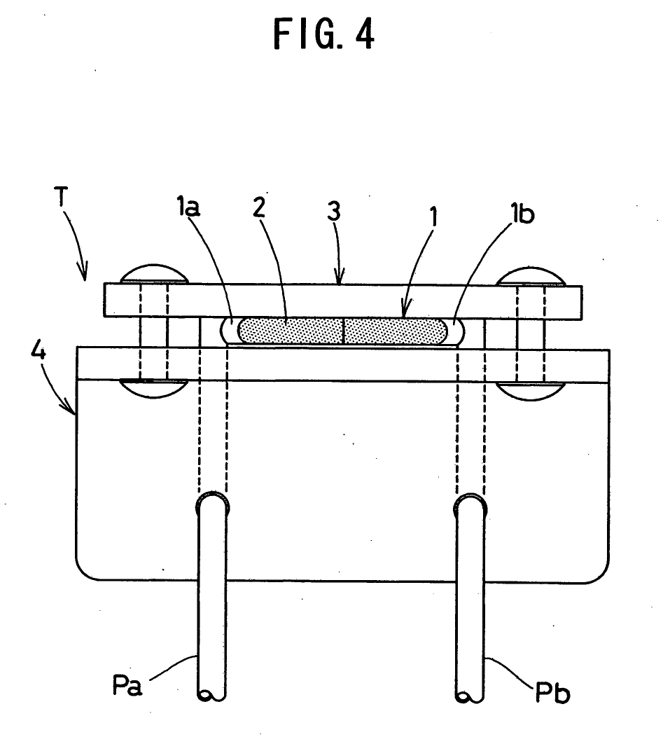

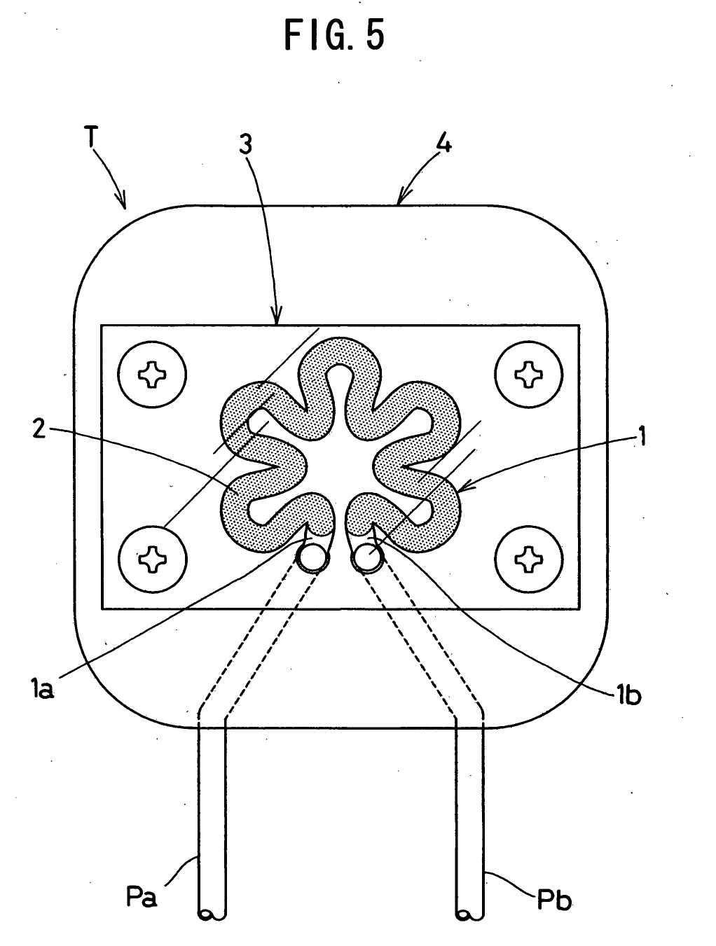

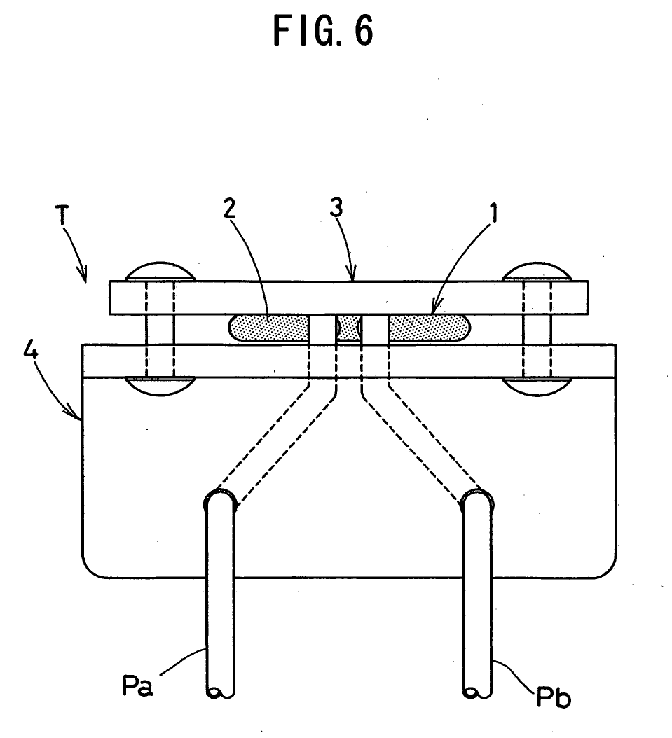

Furthermore, in the chemiluminescence chip according to the present invention, when the filling unit 1 is formed in the shape of a zigzag as shown in FIGS. 3 and 4, or is formed in the shape of a serpentine as shown in FIGS. 5 and 6, it is possible to increase the filling amount of the immobilized enzyme 2. Consequently, labeling enzyme of the material to be measured is more reliably accomplished, and therefore, it is possible to more easily detect and quantify the material to be measured.

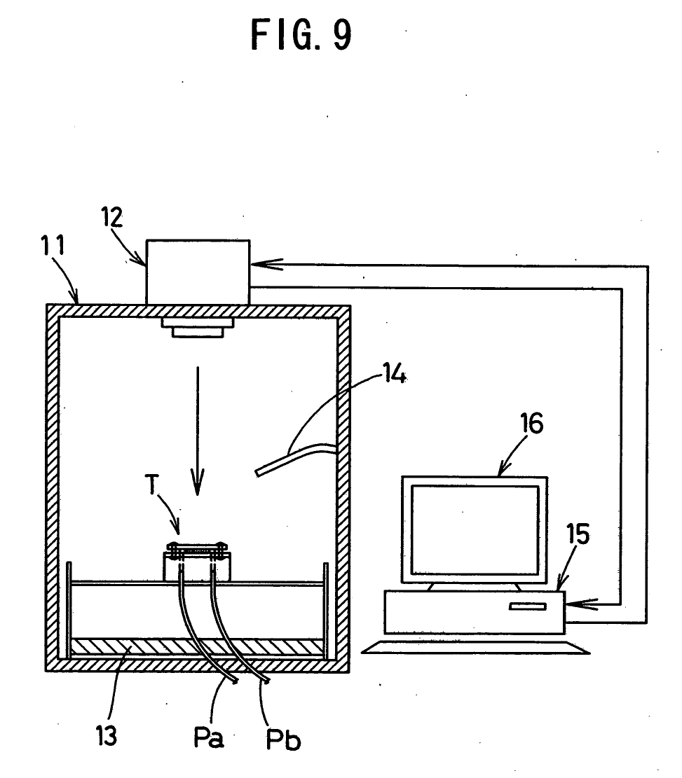

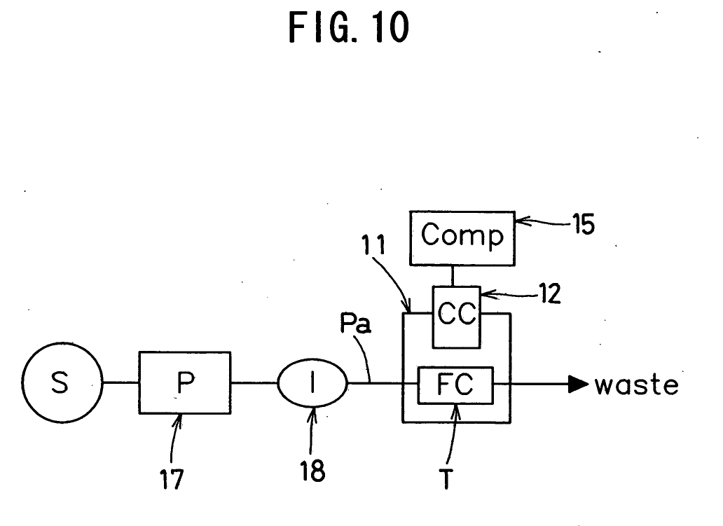

As shown in FIGS. 9 and 10, the chemiluminescence detecting apparatus according to the present invention is constructed such that the above-described chemiluminescence chip T is mounted in a dark box 11, a cooled charge coupled device (CCD) camera 12 is mounted above the chemiluminescence chip T, an inlet pipe Pa, which is disposed outside the dark box 11, is connected to the inlet port 1a of the filling unit 1 of the chemiluminescence chip T, and an outlet pipe Pb, which is also disposed outside the dark box 11, is connected to the outlet port 1b of the filling unit 1.

Furthermore, the chemiluminescence detecting apparatus according to the present invention includes a heater 13 mounted in the dark box 11 for controlling the interior temperature of the dark box 11, and a temperature sensor 14 mounted in the dark box 11 for detecting the interior temperature of the dark box 11. Also, a computer 15 for processing image data photographed by the cooled CCD camera 12, is disposed outside the dark box 11, and a monitor 16 for displaying the image data processed by the computer 15 is also disposed outside the dark box 11. The material to be measured is repeatedly injected into the inlet pipe Pa by means of a liquid-forwarding pump 17 and a loop injector 18.

Hereinafter, the detailed construction of the chemiluminescence chip according to the present invention and the chemiluminescence detecting apparatus using the same will be described in the case that the material to be measured is hydrogen peroxide.

First, the chemiluminescence chip according to the present invention is constructed such that the filling unit 1 is formed of a tube, and the immobilized enzyme 2, which is obtained by immobilizing horseradish peroxidase (hereinafter, referred to as “HRP”) as enzyme, is filled in the tube, the tube is pressed against the supporting structure by the transparent plate 3.

A Teflon tube was used as the tube of the filling unit 1. Kitopal beads (a product from Fuzibo corporation) and imperforate glass beads containing amino group (a product from Kemco corporation) were used as the immobilized enzyme 2.

In the chemiluminescence detecting apparatus according to the present invention, an imidazole solution, pH of which has been adjusted by an alkali buffer solution, is introduced into the inlet pipe Pa by means of the liquid-forwarding pump 17, and an oxygenated water, which has been prepared with various concentrations, is injected into the inlet pipe Pa by means of the loop injector 18. Subsequently, the mixture of the imidazole solution and the oxygenated water flows along the inlet pipe Pa, and reaches the immobilized enzyme 2 of the filling unit 1 through the inlet port 1a of the filling unit 1 of the chemiluminescence chip T. As a result, chemiluminescence is generated in the immobilized enzyme 2. This chemiluminescence is photographed by the cooled CCD camera 12. The photographed image data is processed by the computer 15, and the processed image data is displayed on the monitor 16.

A tricine buffer solution, a tris hydrochloric acid buffer solution, or a boric acid buffer solution may be used as the alkali buffer solution. However, the alkali buffer solution usable in the present invention is not restricted to the above-specified buffer solutions. In this example, tricine buffer solution was used, and the concentration of the tricine buffer solution was 50 mmol/L. In this case, the value of pH was 9.4.

The imidazole usable in the present invention may be imidazole, 2-methylimidazole, 4-methylimidazole, 4-methyl-5-hydroxymethyl imidazole, allantoin, ethylene urea, histidine, pyrazole. However, the imidazole usable in the present invention is not restricted to the above-specified imidazoles. In this example, imidazole was used, and the concentration of the imidazole was 100 mmol/L.

Three kinds of oxygenated water, the concentrations of which are 9.8 μmol/L, 98 μmol/L, and 980 μmol/L, were prepared. Also, the amount of the oxygenated water used was approximately 5 to 50 μL, which is a small amount. In this example, the amount of the oxygenated water used was 20 μL.

Also, it is preferable that the flow speed of the imidazole in the inlet pipe Pa be low. In this example, the flow speed of the imidazole was 100 μL/min. However, it is also possible that the flow speed of the imidazole is less than 100 μL/min.

Furthermore, chemiluminescence is generated by the reaction of the immobilized HRP as the immobilized enzyme 2, imidazole, and oxygenated water, and, after the chemiluminescence is generated, the active center of the HRP is protonized, and therefore, the HRP is inactivated. However, the proton of the active center of the inactivated HRP is removed by the alkali buffer solution and the imidazole. As a result, the HRP is changed into an activated HRP, and therefore, the chemiluminescence is generated again.

As apparent from the above description, in the chemiluminescence detecting apparatus according to the present invention, when the material to be measured is hydrogen peroxide, the chemiluminescence image having a strength corresponding to the concentration of oxygenated water is displayed on the monitor 16.

Consequently, it is possible to easily and conveniently detect and quantify hydrogen peroxide in body fluids, such as sweat, tears, blood, urine, spit, and lymph, by selecting the body fluids as the hydrogen peroxide measuring sample. The detection and quantification of the hydrogen peroxide in the body fluids are necessary to measure oxidative stress and the substrate of oxidase enzyme, and are very useful in a clinical testing field.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A chemiluminescence chip constructed such that a filling unit, in which an immobilized enzyme is filled, is mounted in a space having an inlet port, through which a material to be measured is introduced, and an outlet port, through which the material to be measured is discharged, on a supporting structure

2. The chemiluminescence chip as set forth in claim 1, wherein the filling unit is a transparent tube.

3. The chemiluminescence chip as set forth in claim 1, wherein the filling unit is a slit formed in a synthetic resin sheet.

4. The chemiluminescence chip as set forth in claim 1, wherein the filling unit is a groove formed in a flat plate.

5. The chemiluminescence chip as set forth in claim 1, wherein the filling unit is formed in the shape of a straight line.

6. The chemiluminescence chip as set forth in claim 1, wherein the filling unit is formed in the shape of a zigzag.

7. The chemiluminescence chip as set forth in claim 1, wherein the filling unit is formed in the shape of a serpentine.

8. A chemiluminescence detecting apparatus constructed such that the chemiluminescence chip as set forth in claim 1 is mounted in a dark box, a cooled CCD camera is mounted above the chemiluminescence chip, an inlet pipe, which is disposed outside the dark box, is connected to the inlet port of the filling unit of the chemiluminescence chip, and an outlet pipe, which is also disposed outside the dark box, is connected to the outlet port of the filling unit.

9. The chemiluminescence detecting apparatus as set forth in claim 8, wherein the dark box has a heater and a temperature sensor mounted therein.
